(19) United States
(12) Patent Application Publication
(10) Pub. No.: US 2023/0338355 A1
(43) Pub. Date: Oct. 26, 2023

(54) NK ANTAGONISTS FOR CONTRACEPTION

(71) Applicant: Acer Therapeutics Inc., Newton, MA (US)

(72) Inventor: D. Christopher SCHELLING, Bend, OR (US)

(21) Appl. No.: 18/018,525

(22) PCT Filed: Jul. 30, 2021
(86) PCT No.: PCT/US2021/044018

§ 371 Date: Jan. 27, 2023

Related U.S. Application Data
(60) Provisional application No. 63/059,062, filed on Jul. 30, 2020.

(51) Int. Cl. A61K 31/4545; A61P 15/16; A61P 15/18

(52) U.S. Cl. CPC A61K 31/4545; A61P 15/16; A61P 15/18

(57) ABSTRACT

The present disclosure relates generally to a method for preventing pregnancy in a female patient and to a method of chemically castrating a male patient.

### CROSS REFERENCE TO RELATED APPLICATIONS

This application claims the benefit of priority under 35 U.S.C. §119(e) to U.S. Provisional Application No. 63/059,062, filed Jul. 30, 2020, which is incorporated herein by reference in its entirety.

### BACKGROUND

Contraception is a means of preventing pregnancy by implementing a method or implanting a device. Hormonal contraception is a widely used and generally successful means of preventing pregnancy. The administration of either progestin or estrogen-progestin combinations to female patients, via contraceptive pills, implants, injections, patches, intrauterine devices, or vaginal rings, prevents pregnancy through two main routes. Hormonal contraception increases the viscosity of cervical mucus, inhibiting sperm motility and viability, thereby limiting access to the female upper genital tract, and/or prevents ovulation by suppressing the cyclical frequency of gonadotropin-releasing hormone (GnRH), which inhibits follicular development.

While the implementation of a hormonal contraceptive regimen is generally well tolerated, increased incidences of cardiovascular disease, blood clots, and various cancers are known, especially for patients with pre-existing conditions or who are at a heightened risk for these complications. Similarly, the administration of non-hormonal compounds that act on sex hormone production and availability can provide for chemical castration in male patients. In certain instances of hormone-dependent prostate cancer, administration of antiandrogenic drugs to decrease endogenous testosterone levels in the body can aid in treatment. For individuals with a predisposition for developing hormone-sensitive cancers, including certain forms of breast, ovarian, endometrial, prostate, and testicular cancers, the use of hormonal therapeutic agents to prevent pregnancy or to chemically castrate is contraindicated.

Accordingly, a need exists for a non-hormonal therapeutic agent for preventing pregnancy in female patients and chemically castrating male patients.

### SUMMARY

Provided herein is a method for preventing pregnancy in a female patient, comprising administering to said patient, an effective amount of a neurokinin receptor (NK) antagonist. In certain embodiments, the patient has cancer, has had cancer, or is at an increased risk for cancer. In one embodiment, one or more than one NK antagonist is administered. In one embodiment, the NK receptor antagonist is a NK3 antagonist. In one embodiment, the NK antagonist is osanetant, or a stereoisomer, mixture of stereoisomers, prodrug, pharmaceutically acceptable salt, hydrate, solvate, acid salt hydrate, N-oxide or isomorphic crystalline form thereof.

Also provided herein is a method of chemically castrating a male patient, comprising administering to said patient, an effective amount of a neurokinin receptor (NK) antagonist. In certain embodiments, the patient has cancer, has had cancer, or is at an increased risk for cancer. In one embodiment, one or more than one NK antagonist is administered. In one embodiment, the NK receptor antagonist is a NK3 antagonist. In one embodiment, the NK antagonist is osanetant, or a stereoisomer, mixture of stereoisomers, prodrug, pharmaceutically acceptable salt, hydrate, solvate, acid salt hydrate, N-oxide or isomorphic crystalline form thereof.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### Definitions

Unless defined otherwise, all technical and scientific terms used herein have the same meaning as is commonly understood by one of ordinary skill in the art. As used herein, the below terms have the following meanings unless specified otherwise. Any methods, devices and materials similar or equivalent to those described herein can be used in the practice of the compositions and methods described herein. The following definitions are provided to facilitate understanding of certain terms used frequently herein and are not meant to limit the scope of the present disclosure. All references referred to herein are incorporated by reference in their entirety.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, references to “the agent” includes a plurality of such agents.

Reference to “about” a value or parameter herein includes (and describes) embodiments that are directed to that value or parameter per se. In certain embodiments, the term “about” includes the indicated amount ± 10%. In other embodiments, the term “about” includes the indicated amount ± 5%. In certain other embodiments, the term “about” includes the indicated amount ± 1%. Also, to the term “about X” includes description of “X.”

“Pharmaceutically acceptable” or “physiologically acceptable” refer to compounds, salts, compositions, dosage forms and other materials which are useful in preparing a pharmaceutical composition that is suitable for human or veterinary pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropylamine, trimethyl amine, diethyl amine, tri(isopropyl) amine, tri(n-propyl) amine, ethanolamine, 2-dimethylaminoethanol, piperazine, piperidine, morpholine, N-ethylpiperidine, and the like.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposable mirror images of one another.

A “prodrug” is any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein and the like. Preparation, selection and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

As used herein, the term “solvate” refers to a complex formed by combining a compound and a solvent.

As used herein, the term “hydrate” refers to a complex formed by combining a compound and water (i.e., a solvate when the solvent is water).

As used herein, the term “acid salt hydrate” refers to a complex formed by combining an acid salt compound with water.

As used herein, the term “N-oxide” refers to an oxidized tertiary or pyridinyl amine moiety.

As used herein, the term “isomorphic crystalline form” refers to two or more crystalline forms that have the same space group, unit-cell dimensions, and types and positions of atoms, with the exception of a replacement of one or more atoms in one isomorphic crystalline form with a different atom in its counterpart isomorphic crystalline form.

As used herein, the term “administration” refers to introducing an agent into a patient. For example, a therapeutic amount can be administered to the patient, which can be determined by the treating physician, medical professional, or the like. In some embodiments, a therapeutic amount is administered orally. In some embodiments, a therapeutic amount is administered intranasally. In some embodiments, a therapeutic amount is administered subcutaneously. In some embodiments, a therapeutic amount is administered transdermally. In some embodiments, a therapeutic amount is administered intravenously. In some embodiments, a therapeutic amount is administered buccally. The related terms and phrases “administering” and “administration of,” when used in connection with a compound or tablet (and grammatical equivalents) refer both to direct administration, which may be administration to a patient by a medical professional or by self-administration by the patient, and/or to indirect administration, which may be the act of prescribing a drug. Administration entails delivery to the patient of the drug.

The term “dose” or “dosage” refers to the total amount of an active agent (e.g., osanetant or a pharmaceutically acceptable salt thereof) administered to a patient in a single day (24-hour period). The desired dose can be administered once daily. In some embodiments, the desired dose may be administered in one, two, three, four or more sub-doses at appropriate intervals throughout the day, where the cumulative amount of the sub-doses equals the amount of the desired dose administered in a single day. The terms “dose” and “dosage” are used interchangeably herein.

As used herein, “effective amount,” “therapeutically effective amount” or “therapeutic amount” refers to an amount of a drug or an agent (e.g., osanetant or a pharmaceutically acceptable salt thereof) that when administered to a patient suffering from a condition, will have the intended therapeutic effect, e.g., alleviation, amelioration, palliation or elimination of one or more manifestations of the condition in the patient. The full therapeutic effect does not necessarily occur by administration of one dose, and can occur only after administration of a series of doses and can be administered in one dose form or multiples thereof. For example, 500 mg of the drug can be administered in a single 500 mg strength tablet or two 250 mg strength tablets. Thus, a therapeutically effective amount may be administered in one or more administrations.

As used herein, the term “patient” or “subject” refers to a mammal, such as a human, bovine, rat, mouse, dog, monkey, ape, goat, sheep, cow, or deer. A patient as described herein can be a human.

As used herein, “treatment,” “treating,” and “treat” are defined as acting upon a disease, disorder, or condition with an agent to reduce or ameliorate the harmful or any other undesired effects of the disease, disorder, or condition and/or its symptoms. Treatment, as used herein, covers the treatment of a human patient, and includes: (a) reducing the risk of occurrence of the condition in a patient determined to be predisposed to the disease but not yet diagnosed as having the condition, (b) impeding the development of the condition, and/or (c) relieving the condition, i.e., causing regression of the condition and/or relieving one or more symptoms of the condition.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

As used herein, a “hormone contraceptive” is pharmaceutical composition administered to a female patients, containing either progestin or an estrogen-progestin combination, as a pill, implant, injection, patch, intrauterine device, or vaginal ring, which prevents pregnancy. Hormone contraceptives are a widely used and generally successful means of preventing pregnancy.

### Methods

Provided herein are methods for preventing pregnancy in a female patient, comprising administering to said patient, an effective amount of a neurokinin receptor (NK) antagonist. In certain embodiments, the female patient has cancer, has had cancer, or is at an increased risk for cancer. The cancer can be selected from a variety of cancers, including, but not limited to, those cancers discussed below.

In certain embodiments, provided herein are methods for preventing pregnancy in a female patient for whom a hormone-based contraceptive is contraindicated, comprising administering an effective amount of a neurokinin receptor (NK) antagonist. As discussed below, in certain embodiments the NK antagonist may be a neurokinin-1 receptor antagonist, a neurokinin-2 receptor antagonist, a neurokinin-3 receptor antagonist, or a combination of one or more thereof. In certain embodiments, the NK antagonist may be a neurokinin-3 receptor antagonist. In certain embodiments, the neurokinin-3 receptor antagonist is selected from osanetant, fezolinetant, pavinetant, talnetant, SB-222,200, SB-218,795, and NT-814. In certain embodiments, the neurokinin-3 receptor antagonist is osanetant. In certain embodiments, the neurokinin-3 receptor antagonist is fezolinetant. In certain embodiments, the neurokinin-3 receptor antagonist is pavinetant. In certain embodiments, the neurokinin-3 receptor antagonist is talnetant. In certain embodiments, the neurokinin-3 receptor antagonist is SB-222,200. In certain embodiments, the neurokinin-3 receptor antagonist is SB-218,795. In certain embodiments, the neurokinin-3 receptor antagonist is NT-814.

In some embodiments provided herein, the female patient does not have polycystic ovary syndrome, or has not been diagnosed with polycystic ovary syndrome. In some embodiments provided herein, the female patient does not have polycystic ovary syndrome, but is a patient for whom a hormone-based contraceptive is contraindicated.

Also provided herein are methods for preventing pregnancy in a female patient who has a cancer, has had a cancer, or has an increased risk for a cancer, comprising administering an effective amount of osanetant, or a stereoisomer, mixture of stereoisomers, prodrug, pharmaceutically acceptable salt, hydrate, solvate, acid salt hydrate, N-oxide or isomorphic crystalline form thereof. The chemical name of osanetant is (R)-N-(1-(3-(1-benzoyl-3-(3,4-dichlorophenyl)piperidin-3-yl)propyl)-4-phenylpiperidin-4-yl)-N-methylacetamide, and has the following structure:

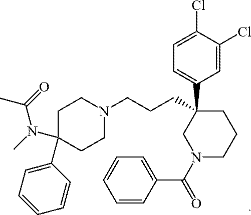

In some embodiments provided herein, the female patient has cancer. In some embodiments, the female patient suffered from cancer but is in remission. In some embodiments, the female patient has an increased risk for cancer. In some embodiments, such a risk may be due to a genetic predisposition to developing certain mutation-linked or hormone-dependent tumors and cancers.

In some embodiments, the cancer is breast cancer. In some embodiments, the cancer is metastatic breast cancer. In some embodiments, the cancer is ovarian cancer. In some embodiments, the cancer is endometrial cancer.

In some embodiments provided herein, the female patient has been diagnosed with or is at risk of suffering a venous thrombosis (e.g. a blood clot), focal migraines (e.g. migraines associated with sensory disturbance, sensory loss, visual disturbance or visual loss), or hormone-dependent cancers (e.g. breast cancer, ovarian cancer, endometrial cancer).

Also provided herein are methods for chemically castrating a male patient, comprising administering to said patient, an effective amount of a neurokinin receptor (NK) antagonist.

In certain embodiments, the male patient has cancer, has had cancer, or is at an increased risk for cancer. The cancer can be selected from a variety of cancers, including, but not limited to, those cancers discussed throughout.

Also provided herein are methods for chemically castrating a male patient who has a cancer, has had a cancer, or has an increased risk for a cancer, comprising administering an effective amount of osanetant, or a stereoisomer, mixture of stereoisomers, prodrug, pharmaceutically acceptable salt, hydrate, solvate, acid salt hydrate, N-oxide or isomorphic crystalline form thereof.

In some embodiments provided herein, the male patient has cancer. In some embodiments, the male patient suffered from cancer but is in remission. In some embodiments, the male patient has an increased risk for cancer. In some embodiments, such a risk may be due to a genetic predisposition to developing certain mutation-linked or hormone-dependent tumors and cancers. In some embodiments provided herein, the male patient has been diagnosed with or is at risk of suffering from a hormone-dependent cancers (e.g. prostate cancer, testicular cancer).

In some embodiments, the cancer is prostate cancer. In some embodiments, the cancer is testicular cancer.

For hormone-dependent cancers, such as breast, ovarian, endometrial, prostate, and testicular cancers, the proliferation of cells is driven by hormone-receptor interactions on cell surfaces. In the presence of these sex hormones, namely estrogen, progesterone and testosterone, the hormone-dependent cells replicate more frequently, increasing the opportunity for genetic errors to occur and accumulate, potentially leading to cancer. Pharmaceutical interventions for the treatment or prevention of hormone-dependent cancers include compounds that inhibit the synthesis of these sex hormones, such as gonadotropin-releasing hormone receptor (GnRH) agonists and antagonists, and compounds which block receptor sites on hormone-dependent cancer cell surfaces, such as selective estrogen-receptor modulators (SERMs) or nonsteroidal antiandrogens (NSAAs).

As used herein, “selective estrogen-receptor modulators” or “SERMs” are a class of drug which have varied estrogenic and antiestrogenic effects on estrogen receptors, depending on the tissue in which the receptors are located. This allows for selective estrogen receptor modulation in certain tissue types by choosing the appropriate SERM.

In certain embodiments, the patient is, or has been, administered a selective estrogen receptor modulator (SERM). Exemplary SERMs include, but are not limited to, anordrin (+mifepristone (Zi Yun)), bazedoxifene (+conjugated estrogens (Duavee)), broparestrol (Acnestrol), clomifene (Clomid), cyclofenil (Sexovid), lasofoxifene (Fablyn), ormeloxifene (Centron, Novex, Novex-DS, Sevista), ospemifene (Osphena; deaminohydroxytoremifene), raloxifene (Evista), tamoxifen (Nolvadex), toremifene (Fareston; 4-chlorotamoxifen), acolbifene, afimoxifene (4-hydroxytamoxifen; metabolite of tamoxifen), elacestrant, enclomifene ((E)-clomifene), endoxifen (4-hydroxy-N-desmethyltamoxifen; metabolite of tamoxifen), zuclomifene ((Z)-clomifene), arzoxifene, brilanestrant, clomifenoxide (clomiphene N-oxide; metabolite of clomifene), droloxifene (3-hydroxytamoxifen), etacstil, fispemifene, (E)-3-[4-[(E)-1-(4-hydroxyphenyl)-2-phenylbut-1-enyl]phenyl]prop-2-enoic acid (GW-7604, structure below) (4-hydroxyetacstil; metabolite of etacstil), idoxifene (pyrrolidino-4-iodotamoxifen), levormeloxifene ((L)-ormeloxifene), miproxifene, nafoxidine, nitromifene (CI-628), 4-(2-ethyl-11-azatricyclo[5.3.1.04,11]undeca-1(10),2,4,6,8-pentaen-3-yl)phenol (NNC 45-0095, structure below), panomifene, pipendoxifene (ERA-923), trioxifene, and zindoxifene (D-16726).

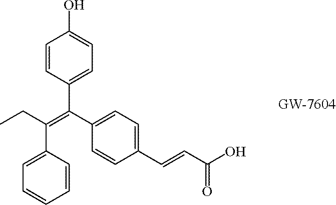 [SMILES: CC/C(=C(\c1ccc(O)cc1)c1ccc(/C=C/C(=O)O)cc1)c1ccccc1]

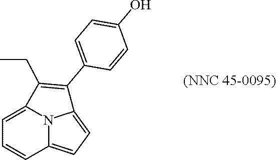 [SMILES: CCc1c(-c2ccc(O)cc2)c2ccc3cccc1n32]

In some embodiments, the patient is, or has been, administered anordrin, bazedoxifene, broparestrol, clomifene, cyclofenil, lasofoxifene, ormeloxifene, ospemifene, raloxifene, tamoxifen, toremifene, acolbifene, afimoxifene, elacestrant, enclomifene, endoxifen, zuclomifene, arzoxifene, brilanestrant, clomifenoxide, droloxifene, etacstil, fispemifene, GW-7604, idoxifene, levormeloxifene, miproxifene, nafoxidine, nitromifene, NNC 45-0095, panomifene, pipendoxifene, trioxifene, or zindoxifene. In some embodiments, the patient is administered anordrin, bazedoxifene, broparestrol, clomifene, cyclofenil, lasofoxifene, ormeloxifene, ospemifene, raloxifene, tamoxifen, toremifene, acolbifene, afimoxifene, elacestrant, enclomifene, endoxifen, zuclomifene, arzoxifene, brilanestrant, clomifenoxide, droloxifene, etacstil, fispemifene, GW-7604, idoxifene, levormeloxifene, miproxifene, nafoxidine, nitromifene, NNC 45-0095, panomifene, pipendoxifene, trioxifene, or zindoxifene. In some embodiments, the patient has not been administered anordrin, bazedoxifene, broparestrol, clomifene, cyclofenil, lasofoxifene, ormeloxifene, ospemifene, raloxifene, tamoxifen, toremifene, acolbifene, afimoxifene, elacestrant, enclomifene, endoxifen, zuclomifene, arzoxifene, brilanestrant, clomifenoxide, droloxifene, etacstil, fispemifene, GW-7604, idoxifene, levormeloxifene, miproxifene, nafoxidine, nitromifene, NNC 45-0095, panomifene, pipendoxifene, trioxifene, or zindoxifene for at least 1 day, at least 2 days, at least 3 days, at least 4 days, or at least 5 days. In some embodiments, the patient has not been administered anordrin, bazedoxifene, broparestrol, clomifene, cyclofenil, lasofoxifene, ormeloxifene, ospemifene, raloxifene, tamoxifen, toremifene, acolbifene, afimoxifene, elacestrant, enclomifene, endoxifen, zuclomifene, arzoxifene, brilanestrant, clomifenoxide, droloxifene, etacstil, fispemifene, GW-7604, idoxifene, levormeloxifene, miproxifene, nafoxidine, nitromifene, NNC 45-0095, panomifene, pipendoxifene, trioxifene, or zindoxifene for at least 1 week.

As used herein, “gonadotropin-releasing hormone receptor agonists and antagonists” or “GnRH receptor agonists and antagonists” are classes of drugs which prevent the GnRH-mediated release of sex hormones.

In certain embodiments, the patient is, or has been, administered a gonadotropin-releasing hormone receptor (GnRH) agonist or antagonist. Exemplary GnRH receptor agonists and antagonists include, but are not limited to, buserelin, deslorelin, fertirelin, gonadorelin, goserelin, histrelin, lecirelin, leuprorelin, nafarelin, peforelin, triptorelin, abarelix, cetrorelix, degarelix, ganirelix, ozarelix, elagolix, linzagolix, opigolix, relugolix, and sufugolix.

As used herein, “nonsteroidal antiandrogens” or “NSAAs” are a class of drug that are antagonists of androgen receptors, blocking the action of testosterone and dihydrotestosterone in tissue. In certain embodiments, the patient is, or has been, administered a nonsteroidal antiangrogen (NSAA). Exemplary NSAAs include, but are not limited to, flutamide, nilutamide, bicalutamide, topilutamide, apalutamide, enzalutamide, darolutamide, cimetidine, proxalutamide, seviteronel, cioteronel, inocoterone acetate, and 4-(3-(4-Hydroxybutyl)-4,4-dimethyl-2,5-dioxoimidazolidin-1-yl)-2-(trifluoromethyl)benzonitrile (RU-58841).

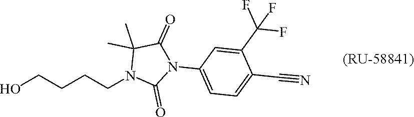 [SMILES: CC1(C)C(=O)N(c2ccc(C#N)c(C(F)(F)F)c2)C(=O)N1CCCCO]

In some embodiments, the female patient is, or has been, administered a hormone or endocrine therapy. In some embodiments, the female patient is administered a hormone or endocrine therapy. In some embodiments, the female patient has not been administered a hormone or endocrine therapy for at least 1 day, at least 2 days, at least 3 days, at least 4 days, or at least 5 days. In some embodiments, the female patient has not been administered a hormone or endocrine therapy for at least 1 week.

Also provided herein are methods for preventing pregnancy in a female patient for which estrogen therapy is contraindicated, comprising administering an effective amount of a neurokinin receptor (NK) antagonist, or a stereoisomer, mixture of stereoisomers, prodrug, pharmaceutically acceptable salt, hydrate, solvate, acid salt hydrate, N-oxide or isomorphic crystalline form thereof. In certain embodiments, the NK antagonist may be a neurokinin-1 receptor antagonist, a neurokinin-2 receptor antagonist, a neurokinin-3 receptor antagonist, or a combination of one or more thereof. In certain embodiments, the NK antagonist may be a neurokinin-3 receptor antagonist. In one embodiment, the NK antagonist is osanetant, or a stereoisomer, mixture of stereoisomers, prodrug, pharmaceutically acceptable salt, hydrate, solvate, acid salt hydrate, N-oxide or isomorphic crystalline form thereof.

### NK Antagonists

As discussed above, NK antagonists are useful in the methods described herein. As used herein, “NK receptor,” “neurokinin receptor,” or “tachykinin receptor” is a transmembrane G-protein coupled receptor. The three known tachykinin receptors are NK1, NK2, and NK3 These receptors act on a variety of human functions, which regulate numerous biological systems, including the reproductive system.

As used herein, “NK receptor antagonists,” “neurokinin receptor antagonists,” or “tachykinin receptor antagonists” are a class of drugs which interact with tachykinin receptors NK1, NK2, and NK3, and dampen the normal agonist-mediated biological responses. The tachykinin receptors have been associated with the transmission of stress signals and pain, the contraction of smooth muscles, inflammation, and modulating the hypothalamus-pituitary-gonadal axis. NK receptor antagonists are indicated for the treatment of migraine, emesis, gastrointestinal disorders, disorders of the reproductive system, and psychiatric disorders, including anxiety, addiction, depression, and schizophrenia. In certain embodiments, the NK antagonist is a NK1, NK2, or NK3 antagonist or a combination thereof.

In certain embodiments, the NK antagonist is a NK1 receptor antagonists in selected from aprepitant, casopitant, ezlopitant, fosaprepitant, lanepitant, maropitant, rolapitant, vestipitant, L-733,060, L-741,671, L-742,694, RP-67580, RPR-100,893, CP-96345, CP-99994, GR-205, 171, TAK-637, T-2328, and combinations thereof. In certain embodiments, the NK antagonist is a NK2 receptor antagonists selected from ibodutant, saredutant, GR-159,897, MEN-10376, and combinations thereof. In certain embodiments, the NK antagonist is a NK3 receptor antagonists selected from fezolinetant, osanetant, pavinetant, talnetant, (S)-3-methyl-2-phenyl-N-(1-phenylpropyl)-4-quinolinecarboxamide (SB-222,200, structure below), (-)-(R)-N-(α-methoxycarbonylbenzyl)-2-phenylquinoline-4-carboxamide (SB-218,795, structure below), and 2-[3,5-bis(trifluoromethyl)phenyl]-N-{4-(4-fluoro-2-methylphenyl)-6-[(7S,9aS)-7-(hydroxymethyl)hexahydropyrazino[2,1-c][1,4]oxazin-8(1H)-yl]pyridin-3-yl}-N,2-dimethylpropanamide (NT-814, structure below)., and combinations thereof.

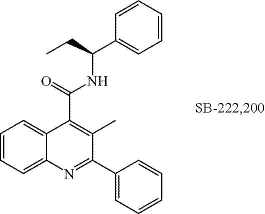 [SMILES: CC[C@H](NC(=O)c1c(C)c(-c2ccccc2)nc2ccccc12)c1ccccc1]

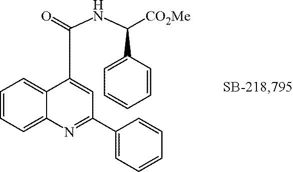 [SMILES: COC(=O)[C@H](NC(=O)c1cc(-c2ccccc2)nc2ccccc12)c1ccccc1]

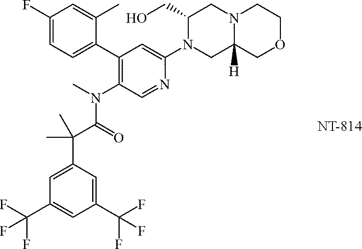 [SMILES: Cc1cc(F)ccc1-c1cc(N2C[C@H]3COCCN3C[C@H]2CO)ncc1N(C)C(=O)C(C)(C)c1cc(C(F)(F)F)cc(C(F)(F)F)c1]

In certain embodiments, the NK antagonist is a NK3 antagonist. The NK3 receptor, and its associated tachykinin neuropeptide, neurokinin B, act on a variety of human functions, affecting the hypothalamus-pituitary-gonadal axis, which regulates numerous biological systems, including the reproductive system. In certain embodiments, the NK3 antagonist is osanetant.

In certain embodiments, the neurokinin-3 receptor antagonist is selected from osanetant, fezolinetant, pavinetant, talnetant, SB-222,200, SB-218,795, and NT-814. In certain embodiments, the neurokinin-3 receptor antagonist is osanetant. In certain embodiments, the neurokinin-3 receptor antagonist is fezolinetant. In certain embodiments, the neurokinin-3 receptor antagonist is pavinetant. In certain embodiments, the neurokinin-3 receptor antagonist is talnetant. In certain embodiments, the neurokinin-3 receptor antagonist is SB-222,200. In certain embodiments, the neurokinin-3 receptor antagonist is SB-218,795. In certain embodiments, the neurokinin-3 receptor antagonist is NT-814. In certain embodiments, the NK3 antagonist is osanetant.

Osanetant was originally developed for the treatment of schizophrenia and other central nervous system disorders. In certain embodiments the NK antagonist is osanetant or a stereoisomer, mixture of stereoisomers, prodrug, pharmaceutically acceptable salt, hydrate, solvate, acid salt hydrate, N-oxide or isomorphic crystalline form thereof. The chemical name of osanetant is (R)-N-(1-(3-(1-benzoyl-3-(3,4-dichlorophenyl)piperidin-3-yl)propyl)-4-phenylpiperidin-4-yl)-N-methylacetamide, and has the following structure:

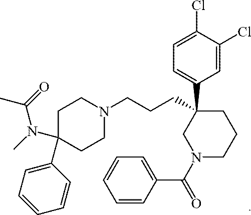

Osanetant can also form pharmaceutically acceptable salts, such as osanetant hydrochloride, osanetant hydrobromide, osanetant sulfate, osanetant hydrogen sulfate, osanetant dihydrogen phosphate, osanetant methanesulfonate, osanetant methyl sulfate, osanetant maleate, osanetant fumarate, osanetant 2-naphthalenesulfonate, osanetant benzenesulfonate, osanetant glycolate, osanetant gluconate, and osanetant citrate, osanetant isethionate, osanetant p-toluenesulfonate, and the like. In some embodiments provided herein, osanetant is administered as a hydrochloride salt thereof.

Osanetant, as well as pharmaceutically acceptable salts thereof, can be purchased from commercial sources or can synthesized using published procedures.

### Administration

In some embodiments, the neurokinin receptor antagonist is orally administered.

In some embodiments, the neurokinin receptor antagonist is intranasally administered.

In some embodiments, the neurokinin receptor antagonist is subcutaneously administered.

In some embodiments, the neurokinin receptor antagonist is transdermally administered.

In some embodiments, the neurokinin receptor antagonist is intravenously administered.

In some embodiments, the neurokinin receptor antagonist is buccally administered.

In some embodiments, the neurokinin receptor antagonist is administered once daily. In some embodiments, the neurokinin receptor antagonist is administered as two, three, four or more sub-doses at appropriate intervals throughout the day, where the cumulative amount of the sub-doses equals the amount of the desired dose administered in a single day.

In some embodiments, when the NK antagonist is osanetant or a stereoisomer, mixture of stereoisomers, prodrug, pharmaceutically acceptable salt, hydrate, solvate, acid salt hydrate, N-oxide or isomorphic crystalline form thereof, the therapeutically effective amount of the osanetant, or a stereoisomer, mixture of stereoisomers, prodrug, pharmaceutically acceptable salt, hydrate, solvate, acid salt hydrate, N-oxide or isomorphic crystalline form thereof, is about 0.25 mg/day to about 1000 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, osanetant is about 0.5 mg/day to about 500 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 0.75 mg/day to about 450 mg/day, is about 1 mg/day to about 400 mg/day or 10 mg/day to about 350 mg/day. In some embodiments, the osanetant is administered is less than about 400 mg/day. In some embodiments, the osanetant is administered is less than about 200 mg/day or about 10 mg/day to about 150 mg/day.

In some embodiments, the therapeutically effective amount of the osanetant, or a stereoisomer, mixture of stereoisomers, prodrug, pharmaceutically acceptable salt, hydrate, solvate, acid salt hydrate, N-oxide or isomorphic crystalline form thereof, is about 1 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 50 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 100 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 200 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 300 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 400 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 500 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 600 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 800 mg/day. In some embodiments, the therapeutically effective amount of osanetant is about 1000 mg/day.

In certain embodiments, osanetant, or a stereoisomer, mixture of stereoisomers, prodrug, pharmaceutically acceptable salt, hydrate, solvate, acid salt hydrate, N-oxide or isomorphic crystalline form thereof, is dosed at about 0.25 mg/day to about 1 mg/day, in order to have desired effect while avoiding transient increases in liver transaminase (alanine aminotransferase) concentrations, and maintaining healthy liver function.

### Pharmaceutical Compositions

Provided herein, in some embodiments, are pharmaceutical compositions that comprise a neurokinin receptor (NK) antagonist, or a stereoisomer, mixture of stereoisomers, prodrug, pharmaceutically acceptable salt, hydrate, solvate, acid salt hydrate, N-oxide or isomorphic crystalline form thereof, and one or more pharmaceutically acceptable vehicles selected from carrier, adjuvants, and excipients. Also provided herein, in some embodiments, are pharmaceutical compositions that comprise osanetant, or a stereoisomer, mixture of stereoisomers, prodrug, pharmaceutically acceptable salt, hydrate, solvate, acid salt hydrate, N-oxide or isomorphic crystalline form thereof, and one or more pharmaceutically acceptable vehicles selected from carrier, adjuvants, and excipients.

Suitable pharmaceutically acceptable vehicles may include, for example, inert solid diluents and fillers, diluents, including sterile aqueous solutions and various organic solvents, permeation enhancers, solubilizers, and adjuvants. Such compositions are prepared in a manner well known in the pharmaceutical art. See, e.g., Remington’s Pharmaceutical Sciences, Mace Publishing Co., Philadelphia, Pa. 17th Ed. (1985); and Modern Pharmaceutics, Marcel Dekker, Inc. 3rd Ed. (G.S. Banker & C.T. Rhodes, Eds.).

The pharmaceutical compositions may be administered in either single or multiple doses. The pharmaceutical composition may be administered by various methods including, for example, rectal, buccal, intranasal, intravenous, subcutaneous, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

The pharmaceutical composition may be in the form of a sterile injectable aqueous or oleaginous suspension. This suspension may be formulated according to the known art using suitable dispersing or wetting agents and suspending agents. The sterile injectable preparation may also be sterile injectable solution or suspension in a non-toxic parentally acceptable vehicle, for example as a solution in 1,3-butanediol. Among the acceptable vehicles that may be employed are water, Ringer’s solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid can be useful in the preparation of injectables. Such solutions may be formulated as 0.01% -10% isotonic solutions, pH 5-7, with appropriate salts.

The compound described herein may be administered parenterally in a sterile medium. Parenteral administration includes subcutaneous injections, intravenous, intramuscular, intrathecal injection or infusion techniques. The compound described herein, depending on the vehicle and concentration used, can either be suspended or dissolved in the vehicle. Advantageously, adjuvants such as local anesthetics, preservatives and buffering agents can be dissolved in the vehicle. In many pharmaceutical compositions for parenteral administration the carrier comprises at least 90% by weight of the total composition. In some embodiments, the carrier for parenteral administration is chosen from propylene glycol, ethyl oleate, pyrrolidone, ethanol, and sesame oil.

A pharmaceutical composition, for example, for injection, may comprise a cyclodextrin. The cyclodextrin may be, for example, a hydroxypropyl cyclodextrin or a sulfobutylether cyclodextrin. The cyclodextrin may be, for example, an α-cyclodextrin, a β-cyclodextrin, or a γ-cyclodextrin.

A compound described herein may also be administered via microspheres, liposomes, other microparticulate delivery systems or sustained release formulations placed in certain tissues including blood. Suitable examples of sustained release carriers include semi-permeable polymer matrices in the form of shared articles, e.g., suppositories or microcapsules. Examples can be found, e.g., in Remington’s Pharmaceutical Sciences, 18th edition, Gennaro, A. R., Lippincott Williams & Wilkins; 20th edition (Dec. 15, 2000) ISBN 0-912734-04-3 and Pharmaceutical Dosage Forms and Drug Delivery Systems; Ansel, N. C. et al. 7th Edition ISBN 0-683305-72-7, the entire disclosures of which are herein incorporated by reference.

Oral administration may be another route for administration of the compounds described herein. Administration may be via, for example, capsule or enteric coated tablets. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat.Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The compound described herein can be incorporated into oral liquid preparations such as aqueous or oily suspensions, solutions, emulsions, syrups, or elixirs, for example. Furthermore, pharmaceutical compositions containing the compound described herein can be presented as a dry product for constitution with water or other suitable vehicle before use. Such liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel, and hydrogenated edible fats), emulsifying agents (e.g., lecithin, sorbitan monooleate, or acacia), non-aqueous vehicles, which can include edible oils (e.g., almond oil, fractionated coconut oil, silyl esters, propylene glycol and ethyl alcohol), and preservatives (e.g., methyl or propyl p-hydroxybenzoate and sorbic acid).

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, orally or nasally, from devices that deliver the formulation in an appropriate manner.

Buccal administration, where the pharmaceutical composition is placed between the gum and cheek and diffuses through the oral mucosa, may be another route for administration of the compounds described herein. The forms in which the pharmaceutical compositions described herein may be incorporated for buccal administration include, for example, quick-dissolving tablets, buccal mucoadhesive tablets, lozenges, powders, sprays, mucoadhesive buccal patches and films, ointments, gels, or liquid suspensions. Formulations for the pharmaceutical compositions for buccal administration that include at least one compound described herein may also include mucoadhesive agents, to maintain prolonged contact of the formulation with the oral mucus membrane, penetration enhancers, to improve drug permeation across the oral mucus membrane, enzyme inhibitors, to protect the active ingredient from enzymatic degradation, and solubility modifiers. The active ingredient is also usually diluted by an excipient.

Some examples of suitable mucoadhesive agents include agarose, chitosan, trimethylated chitosan, chitosan-EDTA, gelatin, hyaluronic acid, guar gum, hakea gum, xanthan gum, gellan gum, carrageenan, pectin, sodium alginate, cellulose derivatives, CMC, thiolated CMC, sodium CMC, HEC, HPC, HPMC, MC, poly(acrylic acid)-based polymers, CP, PC, PAA, copolymers of acrylic acid and PEG, PVA, PVP, CP, aminodextran, dimethylaminoethyl-dextran, hydroxyethyl starch, poly(ethylene oxide), scleroglucan, cyanoacrylate, hydroxylated methacrylate, and poly(methacrylic acid). Some examples of suitable penetration enhancers include sodium lauryl sulfate, cetyl pyridinium chloride, Poloxamer, Brij, Span, Myrj, Tween, sodium glycocholate, sodium tauro deoxycholate, sodium tauro cholate, oleic acid, caprylic acid, lauric acid, lyso phosphatidyl choline, phosphatidyl choline, α-, β-, and γ-cyclodextrin, methylated β-cyclodextrin, EDTA, citric acid, sodium salicylate, methoxy salicylate, chitosan, trimethyl chitosan, poly-L-arginine, and L-lysine. Some examples of suitable enzyme inhibitors include aprotinin, bestatin, and puromycin.

### EXAMPLES

It is understood that modifications which do not substantially affect the activity of the various embodiments of this disclosure are also included within the definition of the disclosure provided herein. Accordingly, the following examples are intended to illustrate but not limit the present disclosure.

### Example 1

Male subjects ages 18-45 are administered osanetant, or a pharmaceutically acceptable salt thereof, or are administered a control (i.e. no treatment), orally and twice daily. Osanetant is administered to the treated group in a total daily dosage of between 1 mg and 400 mg. Blood sampling is performed at various intervals, such as the first and final days of dosing, as well as 0.5, 1, 1.5, 2, 3, 4, 6, 8, 12, 16, and 24 hours postdosing. Blood samples are obtained, for example, to determine levels of serum LH, FSH, testosterone, or plasma osanetant concentration.

### Example 2

Female subjects having regular menstrual cycles (i.e. with an interval of 24-33 days) and having discontinued hormonal contraceptive methods for at least three menstrual cycles before dosing, are administered osanetant, or a pharmaceutically acceptable salt thereof, or are administered a control (i.e. no treatment), orally and twice daily. Osanetant is administered to the treated group in a total daily dosage of between 1 mg and 400 mg. Transvaginal ultrasounds are performed at various intervals, for example, on days 3,8, 13, 18, and 23 to measure follicle diameter and volume, and blood sampling is performed at various intervals, such as the first and final days of dosing, as well as 0.5, 1, 1.5, 2, 3, 4, 6, 8, 12, 16, and 24 hours postdosing. Blood samples are obtained, for example, to determine levels of serum LH, FSH, estradiol, progesterone, or plasma osanetant concentration.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A method for preventing pregnancy in a female patient, comprising administering to said patient, an effective amount of a neurokinin receptor (NK) antagonist.

2. The method of claim 1, wherein the neurokinin receptor antagonist is a neurokinin-3 receptor antagonist.

3. The method of claim 2, wherein the neurokinin-3 receptor antagonist is selected from osanetant, fezolinetant, pavinetant, talnetant, (S)-3-methyl-2-phenyl-N-(1-phenylpropyl)-4-quinolinecarboxamide (SB-222,200), (-)-(R)-N-(α-methoxycarbonylbenzyl)-2-phenylquinoline-4-carboxamide (SB-218,795), and 2-[3,5-bis(trifluoromethyl)phenyl]-N-{4-(4-fluoro-2-methylphenyl)-6-[(7S,9aS)-7-(hydroxymethyl)hexahydropyrazino[2,1-c][1,4]oxazin-8(1H)-yl]pyridin-3-yl}-N,2-dimethylpropanamide (NT-814).

4. The method of claim 3, wherein the neurokinin-3 receptor antagonist is osanetant, or a stereoisomer, mixture of stereoisomers, prodrug, pharmaceutically acceptable salt, hydrate, solvate, acid salt hydrate, N-oxide or isomorphic crystalline form thereof.

5. The method any preceding claim, wherein the patient has cancer, has had cancer, or has an increased risk for cancer.

6. The method of claim 5, wherein the cancer is metastatic breast cancer, ovarian cancer, or endometrial cancer.

7. The method of any one of claims 1-6, wherein the patient is, or has been, administered a hormone or endocrine therapy.

8. The method of any one of claims 4-7, wherein osanetant, or a stereoisomer, mixture of stereoisomers, prodrug, pharmaceutically acceptable salt, hydrate, solvate, acid salt hydrate, N-oxide or isomorphic crystalline form thereof, is administered in a total daily dosage of between 1 mg and 400 mg.

9. The method of claim 8, wherein osanetant, or a stereoisomer, mixture of stereoisomers, prodrug, pharmaceutically acceptable salt, hydrate, solvate, acid salt hydrate, N-oxide or isomorphic crystalline form thereof, is administered orally, intranasally, subcutaneously, intravenously, transdermally, or buccally.

10. A method of chemically castrating a male patient, comprising administering to said patient, an effective amount of a neurokinin receptor (NK).

11. The method of claim 10, wherein the neurokinin receptor antagonist is a neurokinin-3 receptor antagonist.

12. The method of claim 11, wherein the neurokinin-3 receptor antagonist is selected from osanetant, fezolinetant, pavinetant, talnetant, SB-222,200, SB-218,795, and NT-814.

13. The method of claim 12, wherein the neurokinin-3 receptor antagonist is osanetant, or a stereoisomer, mixture of stereoisomers, prodrug, pharmaceutically acceptable salt, hydrate, solvate, acid salt hydrate, N-oxide or isomorphic crystalline form thereof.

14. The method of claim 10, wherein the patient has cancer, has had cancer, or has an increased risk for cancer.

15. The method of claim 14, wherein the cancer is prostate cancer or testicular cancer.

16. The method of any one of claims 13-15, wherein osanetant, or a stereoisomer, mixture of stereoisomers, prodrug, pharmaceutically acceptable salt, hydrate, solvate, acid salt hydrate, N-oxide or isomorphic crystalline form thereof, is administered in a total daily dosage of between 1 mg and 400 mg.

17. The method of claim 16, wherein osanetant, or a stereoisomer, mixture of stereoisomers, prodrug, pharmaceutically acceptable salt, hydrate, solvate, acid salt hydrate, N-oxide or isomorphic crystalline form thereof, is administered orally, intranasally, subcutaneously, intravenously, transdermally, or buccally.

18. The method of any one of claims 1-17, wherein the patient is, or has been, administered anordrin, bazedoxifene, broparestrol, clomifene, cyclofenil, lasofoxifene, ormeloxifene, ospemifene, raloxifene, tamoxifen, toremifene, acolbifene, afimoxifene, elacestrant, enclomifene, endoxifen, zuclomifene, arzoxifene, brilanestrant, clomifenoxide, droloxifene, etacstil, fispemifene, (E)-3-[4-[(E)-1-(4-hydroxyphenyl)-2-phenylbut-1-enyl]phenyl]prop-2-enoic acid (GW-7604), idoxifene, levormeloxifene, miproxifene, nafoxidine, nitromifene, 4-(2-ethyl-11-azatricyclo[5.3.1.04,11]undeca-1(10),2,4,6,8-pentaen-3-yl)phenol (NNC 45-0095), panomifene, pipendoxifene, trioxifene, or zindoxifene.

19. The method of any one of claims 1-17, wherein the patient is, or has been, administered buserelin, deslorelin, fertirelin, gonadorelin, goserelin, histrelin, lecirelin, leuprorelin, nafarelin, peforelin, triptorelin, abarelix, cetrorelix, degarelix, ganirelix, ozarelix, elagolix, linzagolix, opigolix, relugolix, or sufugolix.

20. The method of any one of claims 1-17, wherein the patient is, or has been, administered flutamide, nilutamide, bicalutamide, topilutamide, apalutamide, enzalutamide, darolutamide, cimetidine, proxalutamide, seviteronel, cioteronel, inocoterone acetate, or 4-(3-(4-Hydroxybutyl)-4,4-dimethyl-2,5-dioxoimidazolidin-1-yl)-2-(trifluoromethyl)benzonitrile (RU-58841).
